(19) United States
(12) Patent Application Publication
(10) Pub. No.: US 2019/0083750 A1
(43) Pub. Date: Mar. 21, 2019

(54) CATHETER STEERING ASSEMBLY FOR INTRAVASCULAR CATHETER SYSTEM

(71) Applicant: CRYTERION MEDICAL, INC., Carlsbad, CA (US)

(72) Inventor: David A. Rezac, Westborough, MA (US)

(21) Appl. No.: 16/103,202

(22) Filed: Aug. 14, 2018

Related U.S. Application Data
(60) Provisional application No. 62/560,469, filed on Sep. 19, 2017.

(51) Int. Cl. A61M 25/01; A61B 18/02

(52) U.S. Cl. CPC A61M 25/0147; A61B 2018/00577; A61B 18/02

(57) ABSTRACT

A catheter steering assembly for steering a sheath catheter of an intravascular catheter system includes a first pull wire, a steering member, a first drive member, and a first pulley gear. The first pull wire is connected to the sheath catheter. The steering member rotates about an axis. The first drive member has a first drive member proximal end and a first drive member distal end. The first drive member distal end engages the steering member so that rotation of the steering member rotates the first drive member. The first pulley gear is coupled to the first pull wire. The first pulley gear engages the first drive member proximal end so that rotation of the first drive member rotates the first pulley gear and moves the first pull wire in a direction that is substantially parallel to the axis to articulate the sheath catheter.

### RELATED APPLICATION

This application claims priority on U.S. Provisional Application Ser. No. 62/560,469, filed on Sep. 19, 2017, and entitled “CATHETER STEERING ASSEMBLY FOR AN INTRAVASCULAR CATHETER SYSTEM”. As far as permitted, the contents of U.S. Provisional Application Ser. No. 62/560,469 are incorporated in their entirety herein by reference.

### BACKGROUND

Cardiac arrhythmias involve an abnormality in the electrical conduction of the heart and are a leading cause of stroke, heart disease, and sudden cardiac death. Treatment options for patients with arrhythmias include medications and/or the use of medical devices, which can include implantable devices and/or catheter ablation of cardiac tissue, to name a few. In particular, catheter ablation involves delivering ablative energy to tissue inside the heart to block aberrant electrical activity from depolarizing heart muscle cells out of synchrony with the heart's normal conduction pattern. The procedure is performed by positioning the tip of an energy delivery catheter adjacent to diseased or targeted tissue in the heart. The energy delivery component of the system is typically at or near the most distal (i.e. farthest from the user or operator) portion of the catheter, and often at the tip of the catheter.

Various forms of energy can be used to ablate diseased heart tissue. These can include cryoablation procedures which use cryogenic fluid within cryoballoons (also sometimes referred to herein as “cryogenic balloons” or “balloon catheters”), radio frequency (RF), ultrasound and laser energy, to name a few. During a cryoablation procedure, with the aid of a guide wire, the distal tip of the catheter is positioned adjacent to targeted cardiac tissue, at which time energy is delivered to create tissue necrosis, rendering the ablated tissue incapable of conducting electrical signals. The dose of energy delivered is a critical factor in increasing the likelihood that the treated tissue is permanently incapable of conduction. At the same time, delicate collateral tissue, such as the esophagus, the bronchus, and the phrenic nerve surrounding the ablation zone can be damaged and can lead to undesired complications. Thus, the operator must finely balance delivering therapeutic levels of energy to achieve intended tissue necrosis while avoiding excessive energy leading to collateral tissue injury.

Atrial fibrillation (AF) is one of the most common arrhythmias treated using catheter ablation. AF is typically treated by pulmonary vein isolation, a procedure that removes unusual electrical conductivity in the pulmonary vein. In the earliest stages of the disease, paroxysmal AF, the treatment strategy involves isolating the pulmonary veins from the left atrial chamber. Cryoballoon ablation procedures to treat atrial fibrillation have increased in use in the last several years. In part, this stems from the ease of use, shorter procedure times and improved patient outcomes that are possible through the use of cryoballoon ablation procedures. Despite these advantages, there remains needed improvement to further improve patient outcomes and to better facilitate real-time physiological monitoring of tissue to optimally titrate energy to perform both reversible “ice mapping” and permanent tissue ablation.

The objective of any device for the treatment of AF is to achieve isolation in all, not just some, of the pulmonary veins. Also, it is understood that complete occlusion of each pulmonary vein with the cryogenic balloon is required for adequate antral ablation and electrical isolation. Without pulmonary vein occlusion, blood flow over the balloon during ablation decreases the likelihood of sufficient lesion formation.

As noted, during cryoablation procedures, the catheter is designed to reach tissue within the patient's heart. In order to reach various locations within the heart, the procedure requires that the catheter be carefully steered through the patient's body, particularly through the patient's vascular path. Navigation of the catheter is generally performed with the use of one or more pull wires that typically extend from within a handle assembly and extend distally through a wall of the catheter. Specifically, manipulating the pull wire(s) causes deflection and/or articulation of the catheter, allowing the catheter to be steered and ultimately positioned advantageously in the region of interest, e.g., adjacent to the targeted cardiac tissue, for the cyroablation procedures.

The deflection and/or articulation of the catheter is generally realized by the actuation, i.e., push or pull motion, of the pull wire(s) within the handle assembly. The handle assembly includes a steering member that controls complex configurations of several working components and/or members within the handle assembly in order to achieve the actuation of the pull wire(s). Due to the complex configurations, the steering member generally requires that excessive force and/or rotations be applied. Such complex configurations can also include sequentially nested and threaded components and/or members which make manufacturing inefficient and often cause deficient actuation due to imprecise component interactions. Further, the complex configurations typically require the handle assembly design to be relatively elongated in order to accommodate the working components and/or members.

### SUMMARY

The present invention is directed toward a catheter steering assembly for steering a sheath catheter of an intravascular catheter system. In various embodiments, the catheter steering assembly includes a first pull wire, a steering member, a first drive member, and a first pulley gear. The first pull wire is connected to the sheath catheter. The steering member rotates about an axis. The first drive member has a first drive member proximal end and a first drive member distal end. The first drive member distal end engages the steering member so that rotation of the steering member rotates the first drive member. The first pulley gear is coupled to the first pull wire. The first pulley gear engages the first drive member proximal end so that rotation of the first drive member rotates the first pulley gear and moves the first pull wire in a direction that is substantially parallel to the axis to articulate the sheath catheter.

In some embodiments, the intravascular catheter system includes a sheath catheter handle assembly that is configured to position the sheath catheter during use of the intravascular catheter system. In such embodiments, the steering member can be coupled to at least a portion of the sheath catheter handle assembly.

Additionally, in certain embodiments, the steering member includes internal teeth. In such embodiments, the first drive member distal end is positioned to engage the internal teeth of the steering member so that rotation of the steering member rotates the first drive member.

Further, in some embodiments, the first pulley gear engages the first drive member proximal end so that rotation of the first drive member rotates the first pulley gear about a gear axis that is substantially transverse to the axis.

In certain embodiments, the catheter steering assembly can further include a first pull wire shuttle that is secured to the first pull wire. In some such embodiments, the first pull wire shuttle includes a first pull wire cable that encircles the first pull wire. Further, the first pull wire cable can be connected to the first pulley gear so that as the first pulley gear rotates the first pull wire cable winds around at least a portion of the first pulley gear.

Additionally, in some embodiments, the sheath catheter includes a steering anchor, and the first pull wire can be coupled to the steering anchor.

Further, in certain embodiments, the catheter steering assembly can also include a valve housing that is coupled to a catheter shaft of the intravascular catheter system to isolate the catheter shaft.

In other applications, the present invention is directed toward a catheter steering assembly for steering a sheath catheter of an intravascular catheter system, the catheter steering assembly including a first pull wire that is connected to the sheath catheter; a second pull wire that is connected to the sheath catheter; a steering member that rotates about an axis; a first drive member having a first drive member proximal end and a first drive member distal end, the first drive member distal end engaging the steering member so that rotation of the steering member rotates the first drive member; a second drive member having second drive member proximal end and a second drive member distal end, the second drive member distal end engaging the steering member so that rotation of the steering member rotates the second drive member; a first pulley gear that is coupled to the first pull wire, the first pulley gear engaging the first drive member proximal end so that rotation of the first drive member rotates the first pulley gear and moves the first pull wire in a first direction that is substantially parallel to the axis; and a second pulley gear that is coupled to the second pull wire, the second pulley gear engaging the second drive member proximal end so that rotation of the second drive member rotates the second pulley gear and moves the second pull wire in a second direction that is substantially parallel to the axis; wherein moving of the first pull wire and the second pull wire articulates the sheath catheter.

Additionally, in still other applications, the present invention is further directed toward a catheter steering assembly for steering a sheath catheter of an intravascular catheter system, the catheter steering assembly including a first pull wire that is connected to the sheath catheter; a first pull wire shuttle that is secured to the first pull wire, the first pull wire shuttle including a first pull wire cable that encircles the first pull wire; a second pull wire that is connected to the sheath catheter; a second pull wire shuttle that is secured to the second pull wire, the second pull wire shuttle including a second pull wire cable that encircles the second pull wire; a steering member that rotates about an axis, the steering member including internal teeth; a first drive member having a first drive member proximal end and a first drive member distal end, the first drive member distal end engaging the internal teeth of the steering member so that rotation of the steering member rotates the first drive member; a second drive member having second drive member proximal end and a second drive member distal end, the second drive member distal end engaging the internal teeth of the steering member so that rotation of the steering member rotates the second drive member; a first pulley gear that is coupled to the first pull wire, the first pulley gear engaging the first drive member proximal end so that rotation of the first drive member rotates the first pulley gear about a gear axis that is substantially transverse to the axis and moves the first pull wire in a first direction that is substantially parallel to the axis; and a second pulley gear that is coupled to the second pull wire, the second pulley gear engaging the second drive member proximal end so that rotation of the second drive member rotates the second pulley gear about the gear axis and moves the second pull wire in a second direction that is substantially parallel to the axis; wherein the first pulley gear and the second pulley gear rotate in opposite directions about the gear axis; wherein the first pull wire cable is connected to the first pulley gear so that as the first pulley gear rotates the first pull wire cable winds around at least a portion of the first pulley gear; wherein the second pull wire cable is connected to the second pulley gear so that as the second pulley gear rotates the second pull wire cable winds around at least a portion of the second pulley gear; and wherein moving of the first pull wire and the second pull wire articulates the sheath catheter.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic side view illustration of a patient and one embodiment of an intravascular catheter system having features of the present invention;

FIG. 2 is a simplified schematic side view illustration of a portion of the patient and a portion of an embodiment of the intravascular catheter system including a catheter steering assembly;

FIG. 3 is a simplified schematic top view illustration of a portion of a sheath catheter handle assembly and an embodiment of the catheter steering assembly; and

FIG. 4 is a simplified schematic view illustration of a portion of the sheath catheter handle assembly and another embodiment of the catheter steering assembly.

### DESCRIPTION

Embodiments of the present invention are described herein in the context of a catheter steering assembly for an intravascular catheter system. More specifically, embodiments of the catheter steering assembly, as described in detail herein, are configured to provide high mechanical advantage for the user that overcomes various deficiencies that have been seen with the complex configurations of previous mechanisms for steering the catheter through the patient's body.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound and laser energy, as non-exclusive examples. The present invention is intended to be effective with any or all of these and other forms of energy.

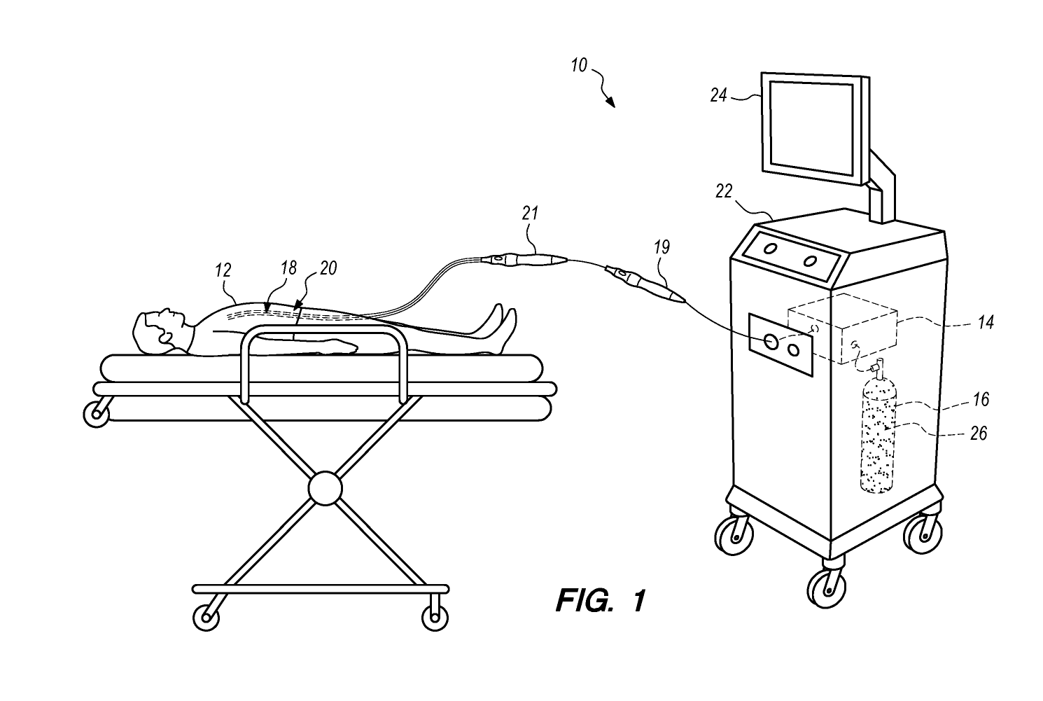

FIG. 1 is a simplified schematic side view illustration of an embodiment of a medical device 10 for use with a patient 12, which can be a human being or an animal. Although the specific medical device 10 illustrated and described herein pertains to and refers to an intravascular catheter system 10 such as a cryogenic balloon catheter system, it is understood and appreciated that other types of medical devices 10 or systems can equally benefit by the teachings provided herein. For example, in certain non-exclusive alternative embodiments, the present invention can be equally applicable for use with any suitable types of ablation systems and/or any suitable types of catheter systems. Thus, the specific reference herein to use as part of an intravascular catheter system is not intended to be limiting in any manner.

The design of the intravascular catheter system 10 can be varied. In certain embodiments, such as the embodiment illustrated in FIG. 1, the intravascular catheter system 10 can include one or more of a control system 14 (illustrated in phantom), a fluid source 16 (illustrated in phantom), a balloon catheter 18, a balloon catheter handle assembly 19, a sheath catheter 20, a sheath catheter handle assembly 21, a control console 22 and a graphical display 24.

It is understood that although FIG. 1 illustrates the structures of the intravascular catheter system 10 in a particular position, sequence and/or order, these structures can be located in any suitably different position, sequence and/or order than that illustrated in FIG. 1. It is also understood that the intravascular catheter system 10 can include fewer or additional components than those specifically illustrated and described herein.

In various embodiments, the control system 14 is configured to monitor and control various processes of the ablation procedure. More specifically, the control system 14 can monitor and control release and/or retrieval of a cooling fluid 26 (e.g., a cryogenic fluid) to and/or from the balloon catheter 18. The control system 14 can also control various structures that are responsible for maintaining and/or adjusting a flow rate and/or pressure of the cryogenic fluid 26 that is released to the balloon catheter 18 during the cryoablation procedure. In such embodiments, the intravascular catheter system 10 delivers ablative energy in the form of cryogenic fluid 26 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18.

Further, or in the alternative, the control system 14 can receive data and/or other information (hereinafter sometimes referred to as “sensor output”) from various structures within the intravascular catheter system 10. In some embodiments, the control system 14 can receive, monitor, assimilate and/or integrate the sensor output and/or any other data or information received from any structure within the intravascular catheter system 10 in order to control the operation of the balloon catheter 18 and/or the sheath catheter 20. As provided herein, in various embodiments, the control system 14 can initiate and/or terminate the flow of cryogenic fluid 26 to the balloon catheter 18 based on the sensor output. Still further, or in the alternative, the control system 14 can control positioning of portions of the balloon catheter 18 and/or the sheath catheter 20 within the body of the patient 12. Yet further, or in the alternative, the control system 14 can control any other suitable functions of the balloon catheter 18 and/or the sheath catheter 20.

The fluid source 16 contains the cryogenic fluid 26, which is delivered to the balloon catheter 18 with or without input from the control system 14 during a cryoablation procedure. Once the ablation procedure has initiated, the cryogenic fluid 26 can be delivered to the balloon catheter 18 and the resulting gas, after a phase change, can be retrieved from the balloon catheter 18, and can either be vented or otherwise discarded as exhaust. Additionally, the type of cryogenic fluid 26 that is used during the cryoablation procedure can vary. In one non-exclusive embodiment, the cryogenic fluid 26 can include liquid nitrous oxide. However, any other suitable cryogenic fluid 26 can be used. For example, in one non-exclusive alternative embodiment, the cryogenic fluid 26 can include liquid nitrogen.

The design of the balloon catheter 18 can be varied to suit the specific design requirements of the intravascular catheter system 10. Additionally, as shown, the balloon catheter 18 is configured to be inserted into the body of the patient 12 during the cryoablation procedure, i.e. during use of the intravascular catheter system 10. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by a healthcare professional (also referred to herein as an “operator”). As used herein, a healthcare professional and/or an operator can include a physician, a physician's assistant, a nurse and/or any other suitable person and/or individual. In certain embodiments, the balloon catheter 18 is positioned within the body of the patient 12 utilizing at least a portion of the sensor output that is received by the control system 14. For example, in various embodiments, the sensor output is received by the control system 14, which can then provide the operator with information regarding the positioning of the balloon catheter 18. Based at least partially on the sensor output feedback received by the control system 14, the operator can adjust the positioning of the balloon catheter 18 within the body of the patient 12 to ensure that the balloon catheter 18 is properly positioned relative to targeted cardiac tissue (not shown). While specific reference is made herein to the balloon catheter 18, as noted above, it is understood that any suitable type of medical device and/or catheter may be used.

The balloon catheter handle assembly 19 is handled and used by the operator to operate, position and control the balloon catheter 18. The design and specific features of the balloon catheter handle assembly 19 can vary to suit the design requirements of the intravascular catheter system 10. In the embodiment illustrated in FIG. 1, the balloon catheter handle assembly 19 is separate from, but in electrical and/or fluid communication with the control system 14, the fluid source 16 and/or the graphical display 24. In some embodiments, the balloon catheter handle assembly 19 can integrate and/or include at least a portion of the control system 14 within an interior of the balloon catheter handle assembly 19. It is understood that the balloon catheter handle assembly 19 can include fewer or additional components than those specifically illustrated and described herein.

In various embodiments, the balloon catheter handle assembly 19 can be used by the operator to initiate and/or terminate the cryoablation process, e.g., to start the flow of the cryogenic fluid 26 to the balloon catheter 18 in order to ablate certain targeted heart tissue of the patient 12. In certain embodiments, the control system 14 can override use of the balloon catheter handle assembly 19 by the operator. Stated in another manner, in some embodiments, based at least in part on the sensor output, the control system 14 can terminate the cryoablation process without the operator using the balloon catheter handle assembly 19 to do so.

The design of the sheath catheter 20 can be varied to suit the specific design requirements of the intravascular catheter system 10. Additionally, as shown, the sheath catheter 20 is configured to be inserted into the body of the patient 12 during the cryoablation procedure, i.e. during use of the intravascular catheter system 10. In one embodiment, at least a portion of the balloon catheter 18 can extend through the sheath catheter 20. Additionally, in one embodiment, the sheath catheter 20 can be positioned within the body of the patient 12 using the control system 14. Stated in another manner, the control system 14 can control positioning of the sheath catheter 20 within the body of the patient 12. Alternatively, the sheath catheter 20 can be manually positioned within the body of the patient 12 by the healthcare professional and/or operator. In certain embodiments, the sheath catheter 20 is positioned within the body of the patient 12 utilizing at least a portion of the sensor output that is received by the control system 14. For example, in various embodiments, the sensor output is received by the control system 14, which then can provide the operator with information regarding the positioning of the sheath catheter 20. Based at least partially on the sensor output feedback received by the control system 14, the operator can adjust the positioning of the sheath catheter 19 within the body of the patient 12. While specific reference is made herein to the sheath catheter 20 as it relates to the intravascular catheter system 10, it is understood that any suitable type of medical device and/or catheter may be used.

The sheath catheter handle assembly 21 is handled and used by the operator to operate, position and control the sheath catheter 20. The design and specific features of the sheath catheter handle assembly 21 can vary to suit the design requirements of the intravascular catheter system 10. In the embodiment illustrated in FIG. 1, the sheath catheter handle assembly 21 is separate from, but in electrical communication with the control system 14 and/or the graphical display 24. In some embodiments, the sheath catheter handle assembly 21 can integrate and/or include at least a portion of the control system 14 within an interior of the sheath catheter handle assembly 21. It is understood that the sheath catheter handle assembly 21 can include fewer or additional components than those specifically illustrated and described herein.

The control console 22 is coupled to the balloon catheter 18, the balloon catheter handle assembly 19, the sheath catheter 20 and the sheath catheter handle assembly 21. Additionally, in the embodiment illustrated in FIG. 1, the control console 22 includes at least a portion of the control system 14, the fluid source 16, and the graphical display 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the graphical display 24.

In various embodiments, the graphical display 24 is electrically connected to the control system 14. Additionally, the graphical display 24 provides the operator of the intravascular catheter system 10 with information and data that can be used before, during and after the cryoablation procedure. For example, the graphical display 24 can provide the operator with information based on the sensor output and any other relevant information that can be used before, during and after the cryoablation procedure. The specifics of the graphical display 24 can vary depending upon the design requirements of the intravascular catheter system 10, or the specific needs, specifications and/or desires of the operator.

In one embodiment, the graphical display 24 can provide static visual data and/or information to the operator. In addition, or in the alternative, the graphical display 24 can provide dynamic visual data and/or information to the operator, such as video data or any other data that changes over time, e.g., during an ablation procedure. Further, in various embodiments, the graphical display 24 can include one or more colors, different sizes, varying brightness, etc., that may act as alerts to the operator. Additionally, or in the alternative, the graphical display 24 can provide audio data or information to the operator.

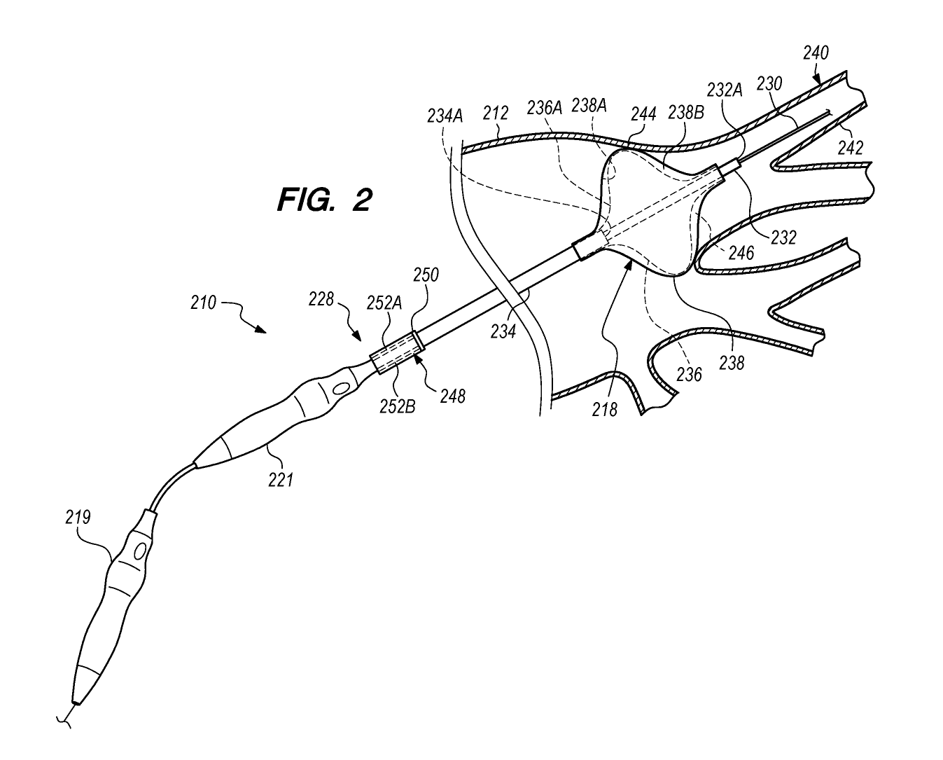

FIG. 2 is a simplified schematic side view illustration of a portion of the patient 212 and a portion of one embodiment of the intravascular catheter system 210. As shown in FIG. 2, in this embodiment, the intravascular catheter system 210 includes one or more of the balloon catheter 218, the balloon catheter handle assembly 219, the sheath catheter 220, and the sheath catheter handle assembly 221. Additionally, as illustrated in FIG. 2, the intravascular catheter system 210 further includes a catheter steering assembly 228. A portion of one embodiment of the catheter steering assembly 228 is shown in FIG. 2. As described herein, in various embodiments, the catheter steering assembly 228 can allow for dual articulation.

The balloon catheter 218 is inserted into the body of the patient 212 during a cryoablation procedure. As noted above, the design of the balloon catheter 218 can be varied to suit the design requirements of the intravascular catheter system 210. In one embodiment, at least a portion of the balloon catheter 218 can extend from the balloon catheter handle assembly 219 and extend through the sheath catheter handle assembly 221 and the sheath catheter 220.

In the embodiment illustrated in FIG. 2, the balloon catheter 218 includes one or more of a guidewire 230, a guidewire lumen 232, a catheter shaft 234, and a balloon assembly 235 including an inner inflatable balloon 236 (sometimes referred to herein as a “first inflatable balloon”, an “inner balloon” or a “first balloon”) and an outer inflatable balloon 238 (sometimes referred to herein as a “second inflatable balloon”, an “outer balloon” or a “second balloon”). As used herein, it is recognized that either balloon 236, 238 can be described as the first balloon or the second balloon. Additionally, it is understood that the balloon catheter 218 can include other structures as well that are not shown and/or described in relation to FIG. 2. However, for the sake of clarity, these other structures have been omitted from the Figures.

As shown in the embodiment illustrated in FIG. 2, the balloon catheter 218 is configured to be positioned within the circulatory system 240 of the patient 212. The guidewire 230 and guidewire lumen 232 are inserted into a pulmonary vein 242 of the patient 212, and the catheter shaft 234 and the balloons 236, 238 are moved along the guidewire 230 and/or the guidewire lumen 232 to near an ostium 244 of the pulmonary vein 242.

In certain embodiments, the guidewire lumen 232 is positioned at least partially within the catheter shaft 234. Additionally, as shown, the guidewire lumen 232 encircles at least a portion of the guidewire 230. During use, the guidewire 230 is inserted into the guidewire lumen 232 and can course through the guidewire lumen 232 and extend out of a distal end 232A of the guidewire lumen 232. In various embodiments, the guidewire 230 can also include a mapping catheter (not shown) that maps electrocardiograms in the heart, and/or can provide information needed to position at least portions of the balloon catheter 218 within the patient 212.

As illustrated in this embodiment, the inner balloon 236 is positioned substantially, if not completely, within the outer balloon 238. With such design, the outer balloon 238 can protect against the cryogenic fluid 26 (illustrated in FIG. 1) leaking out of the balloon assembly 235 should the inner balloon 236 rupture or develop a leak during a cryoablation procedure.

Additionally, in some embodiments, one end of the inner balloon 236 is bonded to a distal end 234A of the catheter shaft 234, and the other end of the inner balloon 236 is bonded near the distal end 232A of the guidewire lumen 232. Further, one end of the outer balloon 238 may be bonded to a neck of the inner balloon 236 or to the distal end 234A of the catheter shaft 234, and the other end of the outer balloon 238 may be bonded to the other end of the inner balloon 236 or to the guidewire lumen 232. Alternatively, the balloons 236, 238 can be secured to other suitable structures. It is appreciated that a variety of bonding techniques can be used and include heat-bonding and adhesive-bonding.

During use, the inner balloon 236 can be partially or fully inflated so that at least a portion of the inner balloon 236 expands toward and/or against at least a portion of the outer balloon 238. Stated in another manner, during use of the balloon catheter 218, at least a portion of an outer surface 236A of the inner balloon 236 expands and can be positioned substantially directly against a portion of an inner surface 238A of the outer balloon 238. As such, when the inner balloon 236 has been fully inflated, the inner balloon 236 and the outer balloon 238 have a somewhat similar physical footprint.

At certain times during usage of the intravascular catheter system 210, the inner balloon 236 and the outer balloon 238 define an inter-balloon space 246, or gap, between the balloons 236, 238. The inter-balloon space 246 is illustrated between the inner balloon 236 and the outer balloon 238 in FIG. 2 for clarity, although it is understood that at certain times during usage of the intravascular catheter system 210, the inter-balloon space 246 has very little or no volume. As provided herein, once the inner balloon 236 is sufficiently inflated, an outer surface 238B of the outer balloon 238 can then be positioned within the circulatory system 240 of the patient 212 to abut and/or substantially form a seal with the ostium 244 of the pulmonary vein 242 to be treated. In particular, during use, it is generally desired that an outer diameter of the balloon assembly 235 be slightly larger than a diameter of the pulmonary vein 242 being treated to best enable occlusion of the pulmonary vein 242. Having a balloon assembly 235 with an outer diameter that is either too small or too large can create problems that inhibit the ability to achieve the desired occlusion of the pulmonary vein 242

The specific design of and materials used for each of the inner inflatable balloon 236 and the outer inflatable balloon 238 can be varied. For example, in various embodiments, specialty polymers with engineered properties can be used for forming the inner inflatable balloon 236. In such embodiments, two specific families of materials can be especially suitable for use in the inner inflatable balloon 236. In particular, some representative materials suitable for the inner inflatable balloon 236 include various grades of polyether block amides (PEBA) such as the commercially available PEBAX® (marketed by Arkema, Colombes, France), or a polyurethane such as Pellathane™ (marketed by Lubrizol). Additionally, or in the alternative, the materials can include PET (polyethylene terephthalate), nylon, polyurethane, and other co-polymers of these materials, as non-exclusive examples. In another embodiment, a polyester block copolymer known in the trade as Hytrel® (DuPont™) is also a suitable material for the inner inflatable balloon 236. Further, the materials may be mixed in varying amounts to fine tune properties of the inner inflatable balloon 236.

Additionally, in certain embodiments, the outer inflatable balloon 238 can be formed from similar materials and can be formed in a similar manner as the inner inflatable balloon 236. For example, some representative materials suitable for the outer inflatable balloon 238 include various grades of polyether block amides (PEBA) such as the commercially available PEBAX®, or a polyurethane such as Pellathane™ Additionally, or in the alternative, the materials can include aliphatic polyether polyurethanes in which carbon atoms are linked in open chains, including paraffins, olefins, and acetylenes. Another suitable material goes by the trade name Tecoflex® (marketed by Lubrizol). Other available polymers from the polyurethane class of thermoplastic polymers with exceptional elongation characteristics are also suitable for use as the outer inflatable balloon 238. Further, the materials may be mixed in varying amounts to fine tune properties of the outer inflatable balloon 238.

The sheath catheter 220 is also inserted into the body of the patient 212 during a cryoablation procedure. As noted above, the design of the sheath catheter 220 can be varied to suit the design requirements of the intravascular catheter system 210. In the embodiment illustrated in FIG. 2, the sheath catheter 220 includes a catheter lumen 248 and a steering anchor 250. Alternatively, the sheath catheter 220 can include additional components or fewer components than those specifically illustrated and described herein. While specific reference is made herein to the sheath catheter 220 including the catheter lumen 248 and/or the steering anchor 250, it is understood that any suitable type of catheter, including the balloon catheter 218, may include the catheter lumen 248 and/or the steering anchor 250.

The catheter lumen 248 is an open space within the interior of the sheath catheter 220. During use, the balloon catheter 218 can be inserted into the catheter lumen 248 and extend through the catheter lumen 248, thereby occupying the open space within the sheath catheter 220 (although a space is shown between the balloon catheter 218 and the sheath catheter 220 in FIG. 2 for clarity).

The steering anchor 250 can be positioned anywhere along the length of the sheath catheter 220. For example, in some embodiments, the steering anchor 250 can be positioned distally from (away from) the sheath catheter handle assembly 221. Alternatively, the steering anchor 250 can be positioned in a different manner from what is specifically shown in FIG. 2.

Additionally, the configuration of the steering anchor 250 can vary. In one non-exclusive embodiment, the steering anchor 250 can have a ring-shaped configuration. In alternative embodiments, the steering anchor 250 can include any other suitable configuration. In certain embodiments, the steering anchor 250 can be connected to the interior of the sheath catheter 220 with the use of an adhesive or a bonding material. Alternatively, the steering anchor 250 may be connected to the interior of the sheath catheter 220 in any suitable manner which may allow the operator to articulate the sheath catheter 220 as desired. Further, the steering anchor 250 may be formed from any suitable material, such as stainless steel or any other suitably rigid material(s).

The catheter steering assembly 228 can allow the sheath catheter 220 to be articulated, steered, guided and/or positioned advantageously during cyroablation procedures. The design of the catheter steering assembly 228 can vary. In the embodiment illustrated in FIG. 2, only a portion of the catheter steering assembly 228, including a first pull wire 252A and a second pull wire 252B, is shown. It is understood that the catheter steering assembly 228 can include additional components than those specifically illustrated and described herein. For example, certain embodiments of the catheter steering assembly 228, and the components thereof, will be described in greater detail herein below. While specific reference is made herein to the sheath catheter 220 as it relates to the catheter steering assembly 228, it is understood that any suitable type of catheter, including the balloon catheter 218, can integrate and/or include the catheter steering assembly 228.

In certain embodiments, the first pull wire 252A can extend generally from within a portion of the sheath catheter handle assembly 221 to the steering anchor 250. Additionally, the second pull wire 252B can also extend generally from within a portion of the sheath catheter handle assembly 221 to the steering anchor 250. In certain embodiments, the pull wires 252A, 252B can be coupled to the sheath catheter handle assembly 221 which can allow the pull wires 252A, 252B to be maneuvered by the operator to articulate, guide and/or position the sheath catheter 220 during cryoablation procedures. The pull wires 252A, 252B can be coupled to the sheath catheter handle assembly 221 in any suitable manner that may allow the operator to articulate, guide and/or position the sheath catheter 220. In various embodiments, the pull wires 252A, 252B can further be coupled to the steering anchor 250 to enable the operator to articulate, steer, navigate or position the sheath catheter 220 as desired. The pull wires 252A, 252B may be coupled to the steering anchor 250 in any suitable manner, i.e., weld or solder joint, adhesive, bonding material, etc.

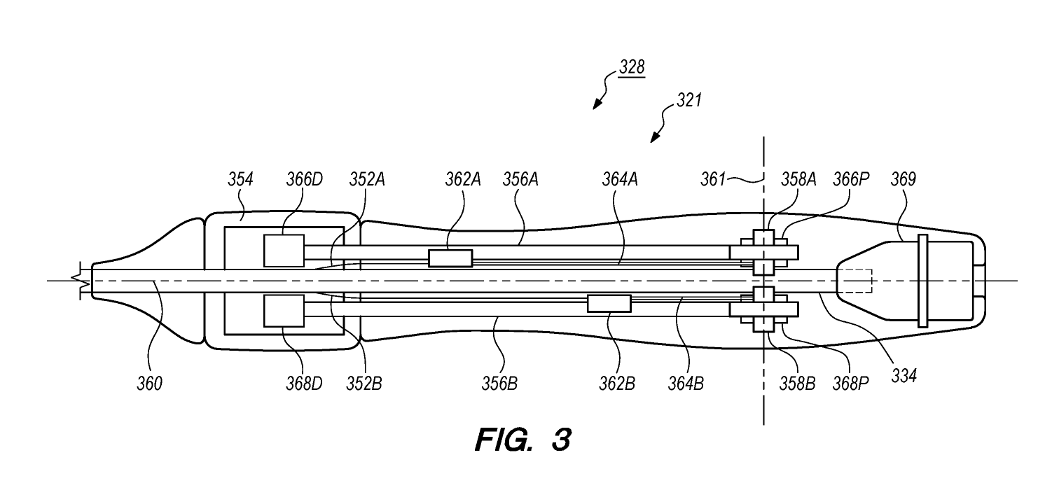

FIG. 3 is a simplified schematic top view illustration of a portion of the sheath catheter handle assembly 321 and an embodiment of the catheter steering assembly 328. In this embodiment, a portion of the sheath catheter handle assembly 321 has been removed so that the various components of the catheter steering assembly 328 are more clearly visible.

In the embodiment illustrated in FIG. 3, the catheter steering assembly 328 can include one or more of the first pull wire 352A, the second pull wire 352B, a steering member 354, a first drive member 356A, a second drive member 356B, a first pulley gear 358A and a second pulley gear 358B. In certain embodiments, such as the embodiment illustrated in FIG. 3, the catheter steering assembly 328 can integrate and/or include at least a portion of the sheath catheter handle assembly 321. In this embodiment, the sheath catheter handle assembly 321 can include a substantially cylindrical design, having an axis 360 (illustrated as a dashed line). In one non-exclusive embodiment, such as shown in FIG. 3, the axis 360 of the sheath catheter handle assembly 321 can be a longitudinal axis. In alternative embodiments, the sheath catheter handle assembly 321 can include any other suitable configuration, shape and/or design. It is understood that the catheter steering assembly 328 can include fewer components or additional components than those specifically illustrated and described herein. For example, in one non-exclusive alternative embodiment, the catheter steering assembly 328 can include only a single pull wire, a single drive member and a single pulley gear. In still other alternative embodiments, the catheter steering assembly 328 can include more than two pull wires, more than two drive members, and more than two pulley gears.

The first pull wire 352A can extend from the sheath catheter handle assembly 321 to the steering anchor 250 (illustrated in FIG. 2), which can be positioned along the length of the sheath catheter 220 (illustrated in FIG. 2). As shown, in certain embodiments, the first pull wire 352A can extend in a direction that is substantially parallel to the longitudinal axis 360 of the sheath catheter handle assembly 321. Similarly, the second pull wire 352B can also extend from the sheath catheter handle assembly 321 to the steering anchor 250, and can also extend in a direction that is substantially parallel to the longitudinal axis 360 of the sheath catheter handle assembly 321. While the embodiment illustrated in FIG. 3 only shows the first and second pull wires 352A, 352B, it is understood that the catheter steering assembly 328 can include more than two or only one pull wires. For example, in certain non-exclusive alternative embodiments, the catheter steering assembly 328 can include one, two, three or four pull wires.

In various embodiments, portions of the pull wires 352A, 352B can be positioned within an interior of the sheath catheter 220. In certain embodiments, the pull wires 352A, 352B can be coupled to the steering anchor 250 to enable the operator to articulate, steer, navigate and/or position the sheath catheter 220. The pull wires 352A, 352B may be coupled to the steering anchor 250 in any suitable manner, i.e., weld or solder joint, press fit, adhesive, bonding material, etc. In certain embodiments, the steering anchor 250 can be connected to the interior of the sheath catheter 220 with the use of an adhesive or a bonding material. Alternatively, the steering anchor 250 may be connected to the interior of the sheath catheter 220 in any other suitable manner.

In certain embodiments, the pull wires 352A, 352B can have a circular cross-section. In alternative embodiments, the cross-section of the pull wires 352A, 352B can have any other suitable shape. Further, the materials from which the pull wires 352A, 352B are formed can include metal or plastic, such as PTFE-coated stainless steel or a para-aramid synthetic fiber, as non-exclusive examples. Alternatively, the pull wires 352A, 352B may be formed from any other suitable material(s).

In some embodiments, the first pull wire 352A can be secured or connected to a first pull wire shuttle 362A. Additionally, the second pull wire 352B can be secured or connected to a second pull wire shuttle 362B. The pull wire shuttles 362A, 362B can act as an intermediate connection which can increase the likelihood of smooth actuation and/or allow the operator to set relative constraints, such as a minimum or maximum degree of movement and/or displacement, for the corresponding pull wires 352A, 352B. Additionally, the pull wire shuttles 362A, 362B can substantially increase the retention strength of the corresponding pull wires 352A, 352B. The configuration of the pull wire shuttles 362A, 362B can vary. In the embodiment illustrated in FIG. 3, the pull wire shuttles 362A, 362B can have a substantially rectangular shape. In alternative embodiments, the pull wire shuttles 362A, 362B can be largely cylindrical, or have any other suitable configuration. Further, in this embodiment, the first pull wire shuttle 362A includes a first pull wire cable 364A, and the second pull wire shuttle 362B includes a second pull wire cable 364B. The pull wire cables 364A, 364B extend from the corresponding pull wire shuttles 362A, 362B and encircle the corresponding pull wires 352A, 352B. Additionally and/or alternatively, the pull wire shuttles 362A, 362B can include additional components or fewer components than those specifically illustrated and described herein. Still alternatively, in other embodiments, the catheter steering assembly 328 can be designed without the pull wire shuttles 362A, 362B. In such embodiments, the pull wires 352A, 352B can be directly connected to cylindrical take-up of the corresponding pulley gear 358A, 358B.

In the embodiment illustrated in FIG. 3, the steering member 354 can rotate about the axis 360. In this embodiment, the steering member 354 can be rotatable about the axis 360 by applying pressure to squeeze the steering member 354 to allow rotation. Alternatively, the steering member can be manipulated in any suitable manner to allow the steering member 354 to rotate about the axis 360.

The design of the steering member 354 can vary. In certain embodiments, the steering member 354 can include a somewhat cylindrical design, i.e., similar to a knob, that is coupled to and/or at least partially surrounds a portion of the sheath catheter handle assembly 321, having substantially the same axis 360. In alternative embodiments, the steering member 354 can include any other suitable shape and/or design. Additionally and/or alternatively, the steering member 354 can be integrated with other structures within the intravascular catheter system 10 (illustrated in FIG. 1).

In various embodiments, the materials from which the steering member 354 is formed can include acrylonitrile butadiene styrene (“ABS”), acetal or polyethylene, as non-exclusive examples. Alternatively, the steering member 354 may be formed from any other suitable material(s).

The first drive member 356A rotates relative to the steering member 354. The second drive member 356B also rotates relative to the steering member 354. The design of the drive members 356A, 356B can vary. In certain embodiments, the first drive member 356A includes a first drive member proximal end 366P and a first drive member distal end 366D. Similarly, the second drive member 356B also includes a second drive member proximal end 368P and a second drive member distal end 368D. In the embodiment illustrated in FIG. 3, the drive member proximal ends 366P, 368P are positioned within the sheath catheter handle assembly 321 away from the steering member 354. The drive member distal ends 366D, 368D are positioned within the sheath catheter handle assembly 321 adjacent to the steering member 354. However, in an alternative embodiment, another suitable configuration can be used.

In certain embodiments, the drive members 356A, 356B can be engaged with the steering member 354, such that as the steering member 354 is manipulated, i.e., rotated about the axis 360, the drive members 356A, 356B can rotate relative to the steering member 354. Stated in another manner, due to the engagement between the drive members 356A, 356B and the steering member 354, rotation of the steering member 354 can result in a corresponding rotation of the drive members 356A, 356B. In some embodiments, the drive members 356A, 356B can rotate about an axis that is substantially parallel to the axis 360. In the embodiment illustrated in FIG. 3, the drive member distal ends 366D, 368D can be engaged with the steering member 354. The drive members 356A, 356B, i.e., via the drive member distal ends 366D, 368D, can be engaged to the steering member 354 in any suitable manner.

In various embodiments, the materials from which the drive members 356A, 356B are formed can include metal or plastic, such as brass, stainless steel, acetal or polyether ether ketone (“PEEK”), as non-exclusive examples. Alternatively, the drive members 356A, 356B may be formed from any other suitable material(s).

In some embodiments, at least a portion of the drive members 356A, 356B can be positioned within at least a portion of the steering member 354 and/or at least a portion of the sheath catheter handle assembly 321. In other embodiments, the drive members 356A, 356B can be positioned solely within the sheath catheter handle assembly 321.

As shown, the first pulley gear 358A is coupled to the first pull wire 352A. Additionally, during use of the catheter steering assembly 328, the first pulley gear 358A can rotate to move, e.g., to wind and/or unwind, the first pull wire 352A. Similarly, the second pulley gear 358B is coupled to the second pull wire 352B. Additionally, during use of the catheter steering assembly 328, the second pulley gear 358B can rotate to move, e.g., to wind and/or unwind, the second pull wire 352B.

The design of the pulley gears 358A, 358B can vary. In certain embodiments, the first pulley gear 358A can be a worm gear that is engaged with the first drive member 356A. In such embodiments, as the steering member 354 is manipulated, i.e. rotated about the axis 360, and the first drive member 356A is rotated relative to the steering member 354 due to its engagement with the steering member 354, the first pulley gear 358A can also be rotated relative to the steering member 354 due to its engagement with the first drive member 356A. In some such embodiments, the first pulley gear 358A can be rotated about a gear axis 361, which can be substantially transverse to the longitudinal axis 360 of the sheath catheter handle assembly 321. Similarly, the second pulley gear 358B can be a worm gear that is engaged with the second drive member 356B. In such embodiments, as the steering member 354 is manipulated, i.e. rotated about the axis 360, and the second drive member 356B is rotated relative to the steering member 354 due to its engagement with the steering member 354, the second pulley gear 358B can also be rotated relative to the steering member 354 due to its engagement with the second drive member 356B. In some such embodiments, the second pulley gear 358B can be rotated about the gear axis 361, which, as noted, can be substantially transverse to the longitudinal axis 360 of the sheath catheter handle assembly 321.

In the embodiment illustrated in FIG. 3, the pulley gears 358A, 358B are engaged with the drive member proximal ends 366P, 368P. The pulley gears 358A, 358B can be engaged with the drive members 356A, 356B, i.e., via the drive member proximal ends 366P, 368P, in any suitable manner.

In various embodiments, as noted above, the first pull wire 352A can be secured and/or connected to the first pulley gear 358A. Similarly, the second pull wire 352B can be secured and/or connected to the second pulley gear 358B. In the embodiment illustrated in FIG. 3, the first pull wire cable 364A, which encircles the first pull wire 352A, is secured and/or connected to the first pulley gear 358A, and the second pull wire cable 364B, which encircles the second pull wire 352B, is secured and/or connected to the second pulley gear 358B. The pull wire cables 364A, 364B can be secured and/or connected to the pulley gears 358A, 358B at any location and/or position. Additionally, the pull wire cables 364A, 364B can be secured, coupled and/or connected to the pulley gears 358A, 358B in any suitable manner, i.e., weld or solder joint, adhesive, bonding material, etc.

In some embodiments, the pull wires 352A, 352B can move, wind and/or unwind as the pulley gears 358A, 358B rotate. For example, in FIG. 3, when the steering member 354 is manipulated, i.e., rotated about the axis 360, the drive members 356A, 356B can then rotate relative to the steering member 354, e.g., about an axis that is substantially parallel to the axis 360. The rotation of the drive members 356A, 356B, in turn can allow the pulley gears 358A, 358B to rotate relative to the drive members 356A, 356B and the steering member 354, e.g., about the gear axis 361. The rotation of the pulley gears 358A, 358B can in turn allow the pull wire shuttles 362A, 362B, including the pull wire cables 364A, 364B, and the pull wires 352A, 352B, to be moved, wound and/or unwound. In certain embodiments, such movement of the pull wires 352A, 352B can include the pull wires 352A, 352B being wound and/or unwound around at least a portion of the corresponding pulley gear 358A, 358B.

The movement, winding and/or unwinding of the pull wire shuttles 362A, 362B, including the pull wire cables 364A, 364B, and the pull wires 352A, 352B, can function to provide articulation of the sheath catheter 220, i.e., to simultaneously push or loosen the first pull wire 352A while pulling or tightening the second pull wire 352B, or vice versa. In other words, when the operator manipulates and/or rotates the steering member 354, the sheath catheter 220 may then be articulated, steered, guided and/or positioned bi-directionally depending on the force being exerted by the first pull wire 352A and/or second pull wire 352B. In various embodiments, the movement of the pull wires 352A, 352B due to such manipulation of the steering member 354 (and the corresponding rotation of the drive members 356A, 356B and the pulley gears 358A, 358B) can be in a direction that is substantially parallel to the axis 360 in order to articulate the sheath catheter 220 as desired.

In certain embodiments, the materials from which the pulley gears 358A, 358B are formed can include metal or plastic, such as brass, stainless steel, acetal or PEEK, as non-exclusive examples. Alternatively, the pulley gears 358A, 358B may be formed from any other suitable material(s).

In certain embodiments, such as the embodiment illustrated in FIG. 3, the catheter steering assembly 328 can further comprise a valve housing 369 that can be coupled to the catheter shaft 334. The valve housing 369 can function to isolate the catheter shaft 334 via a hemostasis valve (not shown), which can be contained within the valve housing 369. The design of the valve housing 369 can vary. The hemostasis valve can be designed to seal a range of catheter shaft sizes during the cryoablation procedure in order to decrease the likelihood of the ingress of air into the balloon catheter 218 (illustrated in FIG. 2) and to decrease the likelihood of blood leaking out of the balloon catheter 218.

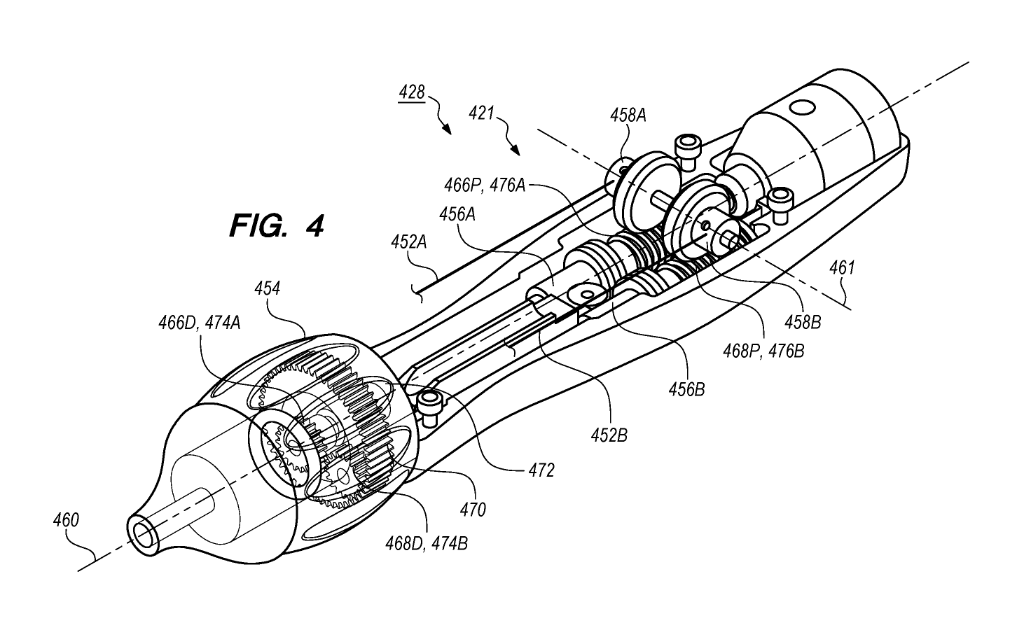

FIG. 4 is a simplified schematic view illustration of a portion of the sheath catheter handle assembly 421 and another embodiment of the catheter steering assembly 428. In the embodiment illustrated in FIG. 4, the catheter steering assembly 428 can include one or more of the first pull wire 452A, the second pull wire 452B, the steering member 454, the first drive member 456A, the second drive member 456B, the first pulley gear 458A and the second pulley gear 458B, which are substantially the same structures and operate in substantially the same manner as described with respect to FIG. 3.

In certain embodiments, the steering member 454 can include internal teeth 470. The design of the internal teeth 470 can vary. For example, the internal teeth 470 can include helical grooves, spur grooves, etc. In the embodiment illustrated in FIG. 4, the internal teeth 470 have spur grooves. In alternative embodiments, the internal teeth 470 can have any other threaded and/or grooved design. Further, in FIG. 4, the steering member 454 can have external grooves 472 to allow the operator to grip, squeeze and/or rotate the steering member 454. While the design of the external grooves 472 in FIG. 4 are substantially oval, it is understood that the shape of the external grooves 472 can vary.

The first drive member 456A includes the first drive member proximal end 466P and the first drive member distal end 466D. Similarly, the second drive member 456B includes the second drive member proximal end 468P and the second drive member distal end 468D. In the embodiment illustrated in FIG. 4, the first drive member distal end 466D can include and/or be coupled to a first drive member gear 474A and the second drive member distal end 468D can include and/or be coupled to a second drive member gear 474B. The drive member distal ends 466D, 468D can be coupled to the drive member gears 474A, 474B in any suitable manner. The design of the drive member gears 474A, 474B can vary. In FIG. 4, the drive member gears 474A, 474B have a spur design. In alternative embodiments, the drive member gears 474A, 474B can have any other suitable design and/or shape. The drive member gears 474A, 474B can be engaged with the internal teeth 470 of the steering member 454, such that as the steering member 454 is manipulated, i.e., rotated about the axis 460 (illustrated by the dashed line), the drive member gears 474A, 474B and the corresponding drive members 456A, 456B can rotate relative to the steering member 454. Additionally and/or alternatively, the drive member gears 474A, 474B can be engaged to the steering member 454 via any other suitable manner.

In some embodiments, the first drive member proximal end 466P can include and/or be coupled to a first drive member screw portion 476A. Similarly, the second drive member proximal end 468P can also include and/or be coupled to a second drive member screw portion 476B. The drive member proximal ends 466P, 468P can be coupled to the drive member screw portions 476A, 476B in any suitable manner. The design of the drive member screw portions 476A, 476B can vary. In the embodiment illustrated in FIG. 4, the drive member screw portions 476A, 476B have helical grooves. The helical grooves can include one of a left-handed thread or a right-handed thread. However, if the first drive member screw portion 476A has the left-handed thread, then the second drive member screw portion 476B will have the right-handed thread, or vice versa. In other words, the drive member screw portions 476A, 476B will include opposite or contrary threads. In alternative embodiments, the drive member screw portions 476A, 476B can include any other suitable configuration.

In various embodiments, the pulley gears 458A, 458B can have a spur design. In certain embodiments, the first pulley gear 458A can be engaged with the first drive member proximal end 466P and the second pulley gear 458B can be engaged with the second drive member proximal end 468P. In the embodiment illustrated in FIG. 4, the pulley gears 458A, 458B are engaged to the drive member proximal ends 466P, 468P via the drive member screw portions 476A, 476B. Alternatively, the pulley gears 458A, 458B can be engaged to the drive members 456A, 456B, i.e., via the drive member proximal ends 466P, 468P, in any other suitable manner.

In certain embodiments, the pull wires 452A, 452B can be secured and/or connected to the pulley gears 458A, 458B. The pull wires 452A, 452B can be secured and/or connected to the pulley gears 458A, 458B at any location and/or position. Additionally, the pull wires 452A, 452B can be secured, coupled and/or connected to the pulley gears 458A, 458B in any suitable manner, i.e., weld or solder joint, adhesive, bonding material, etc.

In some embodiments, the pull wires 452A, 452B can move in a direction substantially parallel to the axis 460, and wind and/or unwind about the pulley gears 458A, 4588, as the pulley gears 458A, 458B rotate about the gear axis 461. For example, in FIG. 4, when the steering member 454 is manipulated, i.e., rotated about the axis 460, the drive members 456A, 456B can rotate relative to the steering member 454 about an axis that is substantially parallel to the axis 460. In this embodiment, the drive member distal ends 466D, 468D are coupled to the drive member gears 474A, 474B. The drive member gears 474A, 474B are engaged with the internal teeth 470 of the steering member 454, such that the drive members 456A, 456B can rotate relative to the steering member 454. The rotation of the drive members 456A, 456B, in turn can allow the pulley gears 458A, 458B to rotate about the gear axis 461, which can allow the pull wires 452A, 452B to be moved, wound and/or unwound in order to articulate the sheath catheter 220 (illustrated in FIG. 2) as desired.

In this embodiment, the drive member proximal ends 466P, 468P are coupled to the drive member screw portions 476A, 476B, which include the left-handed thread and the right-handed thread. As such, the first pulley gear 458A rotates in a first direction, while the second pulley gear 458B simultaneously rotates in a second direction that is opposite the first direction. In other words, the first pulley gear 458A and second pulley gear 458B rotate simultaneously in opposing directions thereby allowing the first pull wire 452A to wind and the second pull wire 452B to unwind, or vice versa. The movement, winding and/or unwinding of the pull wires 452A, 452B provides articulation of the sheath catheter 220, i.e., to simultaneously unwind or loosen the second pull wire 452B while winding or tightening the first pull wire 452A, or vice versa. In other words, when the operator manipulates and/or rotates the steering member 454, the sheath catheter 220 may then be articulated, steered, guided and/or positioned bi-directionally depending upon the force being exerted by the first pull wire 452A and/or second pull wire 452B.

It is appreciated that the embodiments of the catheter steering assembly described in detail herein enable the realization of one or more certain advantages during the cryoablation procedure. With the various designs illustrated and described herein, the catheter steering assembly can substantially decrease the amount of force and/or degree of rotations to allow actuation of the pull wires. The catheter steering assembly can also substantially decrease the likelihood of back-drive, while also effectively reducing the need of minimum linear stroke. Further, the catheter steering assembly can substantially improve the manufacturing process by reducing the length of the handle assembly and allowing an additional quality control step during manufacturing assembly since the pull wires can be positioned and/or tensioned after all the components and/or members are engaged within the handle assembly.

It is understood that although a number of different embodiments of the catheter steering assembly 228 for the intravascular catheter system 210 have been illustrated and described herein, one or more features of any one embodiment can be combined with one or more features of one or more of the other embodiments, provided that such combination satisfies the intent of the present invention.

While a number of exemplary aspects and embodiments of the catheter steering assembly 228 for the intravascular catheter system 210 have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A catheter steering assembly for steering a sheath catheter of an intravascular catheter system, the catheter steering assembly comprising:
a first pull wire that is connected to the sheath catheter;
a steering member that rotates about an axis;
a first drive member having a first drive member proximal end and a first drive member distal end, the first drive member distal end engaging the steering member so that rotation of the steering member rotates the first drive member; and
a first pulley gear that is coupled to the first pull wire, the first pulley gear engaging the first drive member proximal end so that rotation of the first drive member rotates the first pulley gear and moves the first pull wire in a direction that is substantially parallel to the axis to articulate the sheath catheter.

2. The catheter steering assembly of claim 1 wherein the intravascular catheter system includes a sheath catheter handle assembly that is configured to position the sheath catheter during use of the intravascular catheter system, and wherein the steering member is coupled to at least a portion of the sheath catheter handle assembly.

3. The catheter steering assembly of claim 1 wherein the steering member includes internal teeth, and wherein the first drive member distal end is positioned to engage the internal teeth of the steering member so that rotation of the steering member rotates the first drive member.

4. The catheter steering assembly of claim 1 wherein the first pulley gear engages the first drive member proximal end so that rotation of the first drive member rotates the first pulley gear about a gear axis that is substantially transverse to the axis.

5. The catheter steering assembly of claim 1 further comprising a first pull wire shuttle that is secured to the first pull wire.

6. The catheter steering assembly of claim 5 wherein the first pull wire shuttle includes a first pull wire cable that encircles the first pull wire.

7. The catheter steering assembly of claim 6 wherein the first pull wire cable is connected to the first pulley gear so that as the first pulley gear rotates the first pull wire cable winds around at least a portion of the first pulley gear.

8. The catheter steering assembly of claim 1 wherein the sheath catheter includes a steering anchor, and wherein the first pull wire is coupled to the steering anchor.

9. The catheter steering assembly of claim 1 further comprising a valve housing that is coupled to a catheter shaft of the intravascular catheter system to isolate the catheter shaft.

10. A catheter steering assembly for steering a sheath catheter of an intravascular catheter system, the catheter steering assembly comprising:
a first pull wire that is connected to the sheath catheter;
a second pull wire that is connected to the sheath catheter;
a steering member that rotates about an axis;
a first drive member having a first drive member proximal end and a first drive member distal end, the first drive member distal end engaging the steering member so that rotation of the steering member rotates the first drive member;
a second drive member having second drive member proximal end and a second drive member distal end, the second drive member distal end engaging the steering member so that rotation of the steering member rotates the second drive member;
a first pulley gear that is coupled to the first pull wire, the first pulley gear engaging the first drive member proximal end so that rotation of the first drive member rotates the first pulley gear and moves the first pull wire in a first direction that is substantially parallel to the axis; and
a second pulley gear that is coupled to the second pull wire, the second pulley gear engaging the second drive member proximal end so that rotation of the second drive member rotates the second pulley gear and moves the second pull wire in a second direction that is substantially parallel to the axis; and
wherein moving of the first pull wire and the second pull wire articulates the sheath catheter.

11. The catheter steering assembly of claim 10 wherein the intravascular catheter system includes a sheath catheter handle assembly that is configured to position the sheath catheter during use of the intravascular catheter system, and wherein the steering member is coupled to at least a portion of the sheath catheter handle assembly.

12. The catheter steering assembly of claim 10 wherein the steering member includes internal teeth, wherein the first drive member distal end is positioned to engage the internal teeth of the steering member so that rotation of the steering member rotates the first drive member, and wherein the second drive member distal end is positioned to engage the internal teeth of the steering member so that rotation of the steering member rotates the second drive member.

13. The catheter steering assembly of claim 10 wherein the first pulley gear engages the first drive member proximal end so that rotation of the first drive member rotates the first pulley gear about a gear axis that is substantially transverse to the axis, and wherein the second pulley gear engages the second drive member proximal end so that rotation of the second drive member rotates the second pulley gear about the gear axis.

14. The catheter steering assembly of claim 13 wherein the first pulley gear and the second pulley gear rotate in opposite directions about the gear axis.

15. The catheter steering assembly of claim 10 further comprising a first pull wire shuttle that is secured to the first pull wire, and a second pull wire shuttle that is secured to the second pull wire.

16. The catheter steering assembly of claim 15 wherein the first pull wire shuttle includes a first pull wire cable that encircles the first pull wire, and wherein the second pull wire shuttle includes a second pull wire cable that encircles the second pull wire.

17. The catheter steering assembly of claim 16 wherein the first pull wire cable is connected to the first pulley gear so that as the first pulley gear rotates the first pull wire cable winds around at least a portion of the first pulley gear, and wherein the second pull wire cable is connected to the second pulley gear so that as the second pulley gear rotates the second pull wire cable winds around at least a portion of the second pulley gear.

18. The catheter steering assembly of claim 10 wherein the sheath catheter includes a steering anchor, and wherein the first pull wire and the second pull wire are coupled to the steering anchor.

19. The catheter steering assembly of claim 10 further comprising a valve housing that is coupled to a catheter shaft of the intravascular catheter system to isolate the catheter shaft.

20. A catheter steering assembly for steering a sheath catheter of an intravascular catheter system, the catheter steering assembly comprising:
a first pull wire that is connected to the sheath catheter;
a first pull wire shuttle that is secured to the first pull wire, the first pull wire shuttle including a first pull wire cable that encircles the first pull wire;
a second pull wire that is connected to the sheath catheter;
a second pull wire shuttle that is secured to the second pull wire, the second pull wire shuttle including a second pull wire cable that encircles the second pull wire;
a steering member that rotates about an axis, the steering member including internal teeth;
a first drive member having a first drive member proximal end and a first drive member distal end, the first drive member distal end engaging the internal teeth of the steering member so that rotation of the steering member rotates the first drive member;
a second drive member having second drive member proximal end and a second drive member distal end, the second drive member distal end engaging the internal teeth of the steering member so that rotation of the steering member rotates the second drive member;
a first pulley gear that is coupled to the first pull wire, the first pulley gear engaging the first drive member proximal end so that rotation of the first drive member rotates the first pulley gear about a gear axis that is substantially transverse to the axis and moves the first pull wire in a first direction that is substantially parallel to the axis; and
a second pulley gear that is coupled to the second pull wire, the second pulley gear engaging the second drive member proximal end so that rotation of the second drive member rotates the second pulley gear about the gear axis and moves the second pull wire in a second direction that is substantially parallel to the axis;
wherein the first pulley gear and the second pulley gear rotate in opposite directions about the gear axis;
wherein the first pull wire cable is connected to the first pulley gear so that as the first pulley gear rotates the first pull wire cable winds around at least a portion of the first pulley gear;
wherein the second pull wire cable is connected to the second pulley gear so that as the second pulley gear rotates the second pull wire cable winds around at least a portion of the second pulley gear; and
wherein moving of the first pull wire and the second pull wire articulates the sheath catheter.
